(19) United States
(12) Patent Application Publication
(10) Pub. No.: US 2008/0177378 A1
(43) Pub. Date: Jul. 24, 2008

(54) PARTIALLY BIOABSORBABLE IMPLANT

(75) Inventor: Soheil Asgari, Wiesbaden (DE)

Correspondence Address: DORSEY & WHITNEY LLP;INTELLECTUAL PROPERTY DEPARTMENT, 250 PARK AVENUE, NEW YORK, NY, 10177, US

(73) Assignee: CIVENTION AG, Wiesbaden (DE)

(21) Appl. No.: 12/016,835

(22) Filed: Jan. 18, 2008

Related U.S. Application Data
(60) Provisional application No. 60/885,715, filed on Jan. 19, 2007.

(51) Int. Cl. A61F 2/82; A61F 2/02

(52) U.S. Cl. 623/1.38; 424/426

(57) ABSTRACT

An exemplary embodiment of the present invention provides an at least partially biodegradable medical implant having a plurality of in-vivo biodegradable organic polymer particles embedded in a matrix of a plurality of compressed metal-based particles. Methods for the manufacture thereof are also provided. The implant may preferably be further functionalized by inclusion of active ingredients such as therapeutically active agents or markers.

### CROSS-REFERENCE TO RELATED APPLICATIONS

The present invention claims priority of U.S. provisional application Ser. No. 60/885,715, filed on Jan. 19, 2007, the entire disclosure of which is incorporated herein by reference.

### FIELD OF THE INVENTION

The present invention relates to at least partially bioabsorbable implants and methods for the manufacture thereof which use powder molding techniques.

### BACKGROUND OF THE INVENTION

Implants are widely used as short-term or long-term devices to be implanted into the human body in different fields of applications such as orthopedic, cardiovascular or surgical reconstructive treatments. Typically, implants are made of solid materials, either polymers, ceramics or metals. To provide improvements of engraftment or ingrowth of the surrounding tissue or adhesion, or to enable drug-delivery, implants have also been produced with porous structures. Different methods have been established to obtain either completely porous implants, particularly in the orthopedic field of application, or implants having at least porous surfaces, wherein a drug may be included for in-vivo release.

Powder metallurgy and powder shaping methods have been used for producing implants. For example, U.S. Pat. No. 7,094,371 B2 describes a process for manufacturing porous artificial bone graft made of bioceramics such as hydroxyl apatite by extrusion molding of a slurry comprising ceramic powder, a gas-evolving pore-forming system and an organic binder. U.S. Publication Nos. 2006/0239851 A1 and US 2006/0242813 A1 describe metal or powder injection molding processes for the production of metallic or ceramic parts or implants from injectable mixtures comprising a powder and thermoplastic organic binders such as waxes and polyolefins. These powder injection molding (PIM) or metal injection molding (MIM) processes always include the sequential steps of injection molding a more or less net-shaped green part from the powder/binder mixture, substantially removing the binder to form a brown part, and subsequently sintering the brown part at high temperatures to produce the final product. Porosity may be created in these methods by adding placeholders such as inorganic salts or polymers which have to be removed before sintering.

The metal or ceramic powders used in these conventional PIM or MIM processes typically have particle sizes in the micrometer range, usually from 1 to 300 micrometer. After molding and removal of the binder, the parts made of such microparticles have to be sintered to form a mechanically stable product. Sintering is typically done at a temperature slightly below or close to the melting point of the material and held for a predetermined time, so that the particles may form bonds between each other and the material is densified.

There is an increasing need for porous materials to provide implant functionality with additional properties for drug-release or enhanced biocompatibility or the like. Furthermore, there is an increasing demand for implants that can be partially degraded to allow tissue ingrowth and advanced engraftment. Also, implants with additional diagnostic and/or therapeutic properties are required in the field of oncologic treatment. The requirements for such like implants are increasingly complex, because on the one hand the material properties must meet the mechanical requirements and on the other hand provision of functions such as drug-release requires a significant drug amount to be released and bio-available. Therefore a sufficient compartment or space volume for desorption or deposition of the drug itself must be provided without affecting the constructive properties of an implant, particularly its physical properties.

There may be an additional need for improving the availability of drug by increasing the overall volume of the free space in an implant that can contain the drug, without affecting adversely the design of the device. Current design of drug-eluting stents, for example, is often based on non-porous structures that are coated with a drug-eluting layer, resulting in an increase of the stent strut thickness.

Furthermore, there is a need for porous metal-based materials which may be produced in a cost-efficient manner. The powder- or metal-sintering methods mentioned above are technically and economically complex and costly, particularly because of the sintering step that is generally required.

Moreover, increasingly the use of nanoparticles for uptake into tissues and cells is established allowing for enrichment of particles in tissue for imaging purposes or for therapeutic purposes.

### SUMMARY OF THE INVENTION

It is one object of the present invention to provide an implant capable of releasing active ingredients such as e.g., a drug or a marker, etc. Another object of the present invention is to provide implants with sufficient pore volume, whereby the pore sizes are controllable for incorporating large amounts of active ingredients. Another object of the present invention is to provide an implant that releases nanoparticles for diagnostic or therapeutic purposes, particularly in combination with a second pharmacological or diagnostically active compound or any combination thereof.

Exemplary embodiment of manufacturing methods should include possibilities to accurately control pore sizes, mechanical and dimensional properties, chemical and physical properties as well as simplifying the manufacturing process and reducing manufacturing costs.

One embodiment of the present invention provides an at least partially biodegradable implant, in which a plurality of first particles of at least one in-vivo biodegradable organic polymer, and a plurality of second particles of at least one metal-based material, wherein the first particles can be embedded in a matrix of compressed second particles.

The biodegradable organic polymer particles in such implants can be degraded and/or absorbed, e.g., by body fluids, after implantation in-vivo. After absorption of the biodegradable particles, the remaining implant preferably has a highly porous structure which can allow for engraftment of the surrounding tissue, can reduce thrombogenesis and other incompatibility reactions etc. Furthermore, the biodegradable organic polymer particles may be used as a carrier for active ingredients, such as drugs or markers.

The second metal-based particles may include at least one of a metal, a metal alloy, a metal oxide, a metal carbide, a metal nitride, or a metal-containing semiconductor, and these particles may for example, have an average particle size in the range from about 0.5 nanometer to about 1,000 nanometer.

In one exemplary embodiment of the invention, the implant may be substantially totally biodegradable. In such embodiments, the second, metal-based particles may preferably include a metal or metal alloy that is biodegradable in-vivo.

In another exemplary embodiment of the invention, the implant may comprise inorganic nanoparticles that can be used for diagnostic labelling of tissue, as carriers for drug-delivery and/or as agents for therapeutic treatment, such as thermotherapy.

The implant may be, e.g. one of a vascular endoprosthesis, an intraluminal endoprosthesis, a stent, a coronary stent, a peripheral stent, a surgical or dental or orthopedic implant, an implantable orthopedic fixation aid, an orthopedic bone prosthesis or joint prosthesis, a bone substitute or a vertebral substitute in the thoracic or lumbar region of the spinal column; an artificial heart or a part thereof, an artificial heart valve, a heart pacemaker casing or electrode, a subcutaneous and/or intramuscular implant, an implantable drug-delivery device, a microchip, or implantable surgical needles, screws, nails, clips, or staples, or a seed implant or the like.

In a further aspect, the present invention provides a method for the manufacture of an at least partially biodegradable implant. The method preferably includes the steps of providing a suspension comprising a plurality of first particles of at least one in-vivo biodegradable organic polymer; a plurality of second particles of at least one metal-based material; and at least one solvent; wherein the first and second particles can be substantially insoluble in the solvent; and molding the suspension to form an implant comprising the first particles embedded in a matrix of compressed second particles.

In an exemplary embodiment, the method includes molding the suspension by at least one of compacting, injection molding, uniaxial or biaxial pressing, isostatic pressing, slip casting, or extrusion molding, to obtain the partially degradable implant.

One advantage of the methods of the present invention is that sintering steps are not required, i.e., the metal-based particles are not sintered.

### BRIEF DESCRIPTION OF THE DRAWINGS

Further objects, features and advantages of the invention will become apparent from the following detailed description taken in conjunction with the accompanying figures showing illustrative embodiments of the invention, in which:

FIG. 1 is a schematic diagram that illustrates an example of the left hand side a tubular implant (10), and a partial magnification of the structure thereof illustrating an exemplary structure that is composed of a plurality of spherical particles (20) and (30); and

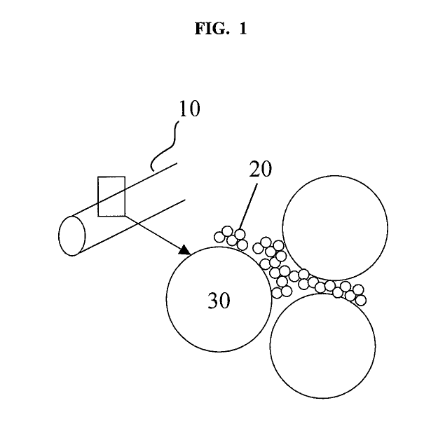

FIG. 2 is a perspective view illustrating an example of the three-dimensional orientation of the spherical particles (20) and (30).

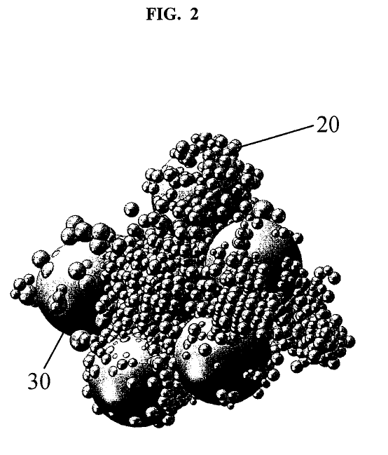

### DETAILED DESCRIPTION OF EXEMPLARY EMBODIMENTS

Without wishing to be bound to any particular theory or exemplary embodiment described herein, it is understood that the molding of suspensions of polymeric particles and metal-based particles under sufficiently high pressures may lead to mechanically stable implantable devices, without the need for sintering steps to be applied or binders to be used. For example, the use of nanoparticles as the metal-based particles instead of conventionally used microparticles can provide sufficient mechanical stability, so that sintering steps can be avoided. By the methods as described herein, at least partially biodegradable implants may be produced in a wide array of desired shapes by compacting suspensions of polymeric particles and metal-based particles to produce the implants in a substantially net-shape. A wide variety of compaction molding procedures may be used.

### Metal-Based Particles

According to the embodiments of the present invention, the basic implant structure can be made from metal-based particles, which can form a matrix into which the biodegradable organic polymer particles are embedded. Typically, the matrix consists of a plurality of discrete metal-based particles, bonded together, e.g., by compression, for adhering the particles to each other. The metal-based particles may be selected from metals or ceramics or any mixture thereof to provide the structural body of the implant.

The metal-based compounds may be, for example, selected from zero-valent metals, metal alloys, shape memory alloys, metal oxides, metal carbides, metal nitrides, and mixed phases thereof such as oxycarbonitrides, oxycarbides etc. These metal-based particles may include those of the main groups of the periodic system of elements, for example, alkaline or alkaline earth metals such as magnesium, calcium, lithium, or transition metals, such as titanium, zirconium, hafnium, vanadium, niobium, tantalum, chromium, molybdenum, tungsten, manganese, rhenium, iron, cobalt, nickel; the noble metals such as gold, silver, ruthenium, rhodium, palladium, osmium, iridium, platinum, copper; or rare earth metals such as e.g., lanthanum, yttrium, cerium, neodymium, samarium, europium, gadolinium, terbium, dysprosium, or holmium. Also stainless steel, memory alloys such as nitinol, nickel titanium alloy, natural or synthetic bone substance, imitation bone based on alkaline earth metal carbonates such as calcium carbonate, magnesium carbonate, strontium carbonate, and any combinations thereof may be used.

In exemplary embodiments of the present invention, the implants may be formed with the use of materials for the metal-based particles, selected from, e.g. stainless steel, platinum-based radiopaque steel alloys, so-called PERSS (platinum-enhanced radiopaque stainless steel alloys), cobalt alloys, titanium alloys, high-melting alloys, e.g., based on niobium, tantalum, tungsten and molybdenum, noble metal alloys, nitinol alloys as well as magnesium alloys and mixtures of the above.

Further suitable exemplary materials for metal-based particles can be Fe-18Cr-14Ni-2.5Mo (“316LVM” ASTM F 138), Fe-21Cr-10Ni-3.5Mn-2.5Mo (ASTM F 1586), Fe-22Cr-13Ni-5Mn (ASTM F 1314), Fe-23Mn-21Cr-1Mo-1N (nickel-free stainless steel); cobalt alloys such as Co-20Cr-15W-10Ni (“L605” ASTM F 90), Co-20Cr-35Ni-10Mo (“MP35N” ASTM F 562), Co-20Cr-16Ni-16Fe-7Mo (“Phynox” ASTM F 1058). Examples of exemplary titanium alloys include CP titanium (ASTM F 67, Grade 1), Ti-6Al-4V (alpha/beta ASTM F 136), Ti-6Al-7Nb (alpha/beta ASTM F1295), Ti-15Mo (beta grade ASTM F 2066); noble metal alloys, such as alloys containing iridium such as Pt-10Ir; nitinol alloys such as martensitic, superelastic and cold-workable (preferably 40%) nitinols and magnesium alloys such as Mg-3Al-1Z.

The metal-based particles can be used in the form of powders, which can be, for example, obtainable by conventional methods such as electrochemical or electrolytical methods, spraying methods such as a rotating electrode process which can lead to spherical particles, or chemical gas phase reduction, flame pyrolysis, plasma methods, high energy milling or precipitation methods.

In exemplary embodiments of the invention, the metal-based particles can have a form as desired, for example selected from spherical particles, dendritic particles, cubes, wires, fibres, tubes and the like. In further exemplary embodiments, the metal-based particles of the above-mentioned materials can include nano- or microcrystalline particles, nanofibers or nanowires. Without wishing to be bound to any particular theory, ultrafine nanosized particles or nanoparticles such as the metal-based particles are particularly useful for manufacturing the implants of the invention.

The metal-based particles useful according to the invention can have an average particle size (D50) from about 0.5 nm to 500 μm, preferably below about 1000 nm, such as from about 0.5 nm to 1,000 nm, or below 900 nm, such as from about 0.5 nm to 900 nm, or from about 0.7 nm to 800 nm.

Preferred D50 particle size distributions can be in a range of about 10 nm up to about 1000 nm, more preferred between 25 nm and 600 nm and most preferred generally falling in the range between 30 nm and 250 nm.

Particle sizes and particle distribution of nanosized particles may be determined by spectroscopic methods such as photocorrelation spectroscopy, or by light scattering or laser diffraction techniques.

The metal-based compounds can be encapsulated in or coated on polymer particles in the process of the present invention. The metal-based particles can also comprise mixtures of different metal-based particles, particularly having e.g. different ferromagnetic properties, x-ray absorption properties or the like, in accordance with the desired properties of the implant to be produced. The metal-based particles may be used in the form of powders, sols, colloidal particles, dispersions, or suspensions.

In exemplary embodiments, particularly for implants with magnetic or signaling properties in general, magnetic metals or alloys such as ferrites, e.g. gamma-iron oxide, magnetite or ferrites of Co, Ni, Mn can be selected as at least a part of the metal-based particles used. Materials having signaling properties are those materials which, when implanted into the human or animal body, can produce a signal which can be detectable by imaging methods such as x-ray, nuclear magnetic resonance, szintigraphy, etc.

Also, semiconducting nanoparticles can be used as at least a part of the metal-based particles in some embodiments, such as e.g. semiconductors of groups II-VI, groups III-V, or groups IV of the periodic system. Suitable group II-VI-semiconductors are, for example, MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, HgTe or mixtures thereof. Examples for group III-V semiconductors are GaAs, GaN, GaP, GaSb, InGaAs, InP, InN, InSb, InAs, AlAs, AlP, AlSb, AlS, or mixtures thereof. Examples for group IV semiconductors are germanium, lead and silicon. The semiconductors may also be used in the form of core-shell-particles. Further, combinations of any of the foregoing semiconductors may be used. Also, complex formed metal-based nanoparticles may be used at least as apart of the metal-based particles, for example, are so-called core-shell configurations, as described explicitly by Peng et al., “Epitaxial Growth of Highly Luminescent CdSe/CdS Core/Shell Nanoparticles with Photo stability and Electronic Accessibility”, Journal of the American Chemical Society, (1997) 119:7019-7029. Preferred in some embodiments can be semiconducting nanoparticles selected from those as listed above, having a core with a diameter of about 1 to 30 nm, such as from about 1 to 15 nm, upon which further semiconducting nanoparticles in about 1 to 50 monolayers, such as about 1 to 15 monolayers are crystallized as a shell. Core and shell may be present in nearly any combination of the materials as described above, preferred in some embodiments are CdSe and CdTe as core and CdS and ZnS as in the shell in such particles.

In a further embodiment of the invention, the metal-based particles can be selected due to their absorptive properties for radiation in a wavelength range from gamma radiation up to microwave radiation, or due to their property to emit radiation, particularly in the region of 60 nm or less. By suitably selecting the metal-based particles, the inventive process can lead to the production of implants having non-linear optical properties, for example materials that block IR-radiation of specific wavelengths suitable for marking purposes or for therapeutic implants absorbing radiation, which may be used e.g. in cancer therapy.

In exemplary embodiments, the metal-based particles, their particle sizes and their diameter of core and shell can be selected from photon emitting compounds, such that the emission is in the range from 20 nm to 1000 nm, or selected from a mixture of suitable particles which emit photons of differing wavelengths when exposed to radiation. In an exemplary embodiment, fluorescent metal-based particles are selected which preferably need not be quenched.

In exemplary embodiments the metal-based particles for biomedical applications may be selected from alkaline earth metal oxides or hydroxides such as e.g., magnesium oxide, magnesium hydroxide, calcium oxide, and calcium hydroxide or mixtures thereof, as well as from biodegradable or biocorrosive metals or alloys based on at least one of magnesium or zinc, or an alloy comprising at least one of Mg, Ca, Fe, Zn, Al, W, Ln, Si, or Y. In this exemplary embodiment, the implant may be substantially completely degradable in-vivo. Examples for suitable biodegradable alloys include e.g., magnesium alloys comprising more than 90% of Mg, about 4-5% of Y, and about 1.5-4% of other rare earth metals such as neodymium and optionally minor amounts of Zr; or biocorrosive alloys comprising as a major component tungsten, rhenium, osmium or molybdenum, for example alloyed with cerium, an actinide, iron, tantalum, platinum, gold, gadolinium, yttrium or scandium.

In further embodiments, the metal-based nanoparticles can be selected from ferromagnetic or superparamagnetic metals or metal-alloys, which may be either further modified by coating with silanes or suitable polymers, or which may not be modified, and which can e.g. be applied for interstitial hyperthermia or thermoablation.

### Organic Polymer Particles

The biodegradable organic polymer particles to be embedded in the metal-based particles may have any desired form such as spherical, cubic, dendritic or fibrous particles or any mixture thereof.

The biodegradable polymer particles can be removed in-vivo, such as by biocorrosion or biodegradation. Any polymer particle that can be degraded, absorbed, metabolized, is resorbable in the human or animal body may be used as the biodegradable organic polymer particle in the embodiments of the present invention. As used in this description, the terms biodegradable, bioabsorbable, resorbable, and biocorrodible are meant to encompass materials that are broken down and may be gradually absorbed or eliminated by the body in-vivo, regardless of whether these processes are due to hydrolysis, metabolic processes, bulk or surface erosion.

The biodegradable polymer particles may be combined with substantially non-biodegradable metal-based particles to form permanent implants, that is, implants that remain in the body for an extended period of time, e.g. to fulfill a supporting function.

In other exemplary embodiments, bio-corrodible metal-based particles, preferably magnesium-based as defined above may be used to produce nonpermanent implants, i.e., implants fulfilling a temporary function in the body and which can be substantially completely absorbed or degraded.

Suitable materials for use in the biodegradable organic polymer particles include biodegradable polymers, for example polymers based on lactic acid such as PLA or PGLA or the like, also proteins. Exemplary materials include collagen, albumin, gelatin, hyaluronic acid, starch, cellulose, methyl cellulose, hydroxypropyl cellulose, hydroxypropylmethyl cellulose, carboxymethyl cellulose phthalate, casein, dextran, polysaccharide, fibrinogen, poly(caprolactone) (PCL), poly(D,L-lactide) (PLA), poly(D,L-lactide-co-glycolide), poly(glycolide), poly(hydroxybutylate), poly(alkyl carbonate), poly(orthoester), biodegradable polyesters, polyiminocarbonates, poly(hydroxyvaleric acid), polydioxanone, poly(ethylene terephtalate), poly(malic acid), poly(tartronic acid), biodegradable polyanhydrides, polyphosphazene, poly(amino acid), and copolymers thereof, such as poly(L-lactide-co-trimethylene carbonate) or poly (L-lactide-co-D,L-lactide). In exemplary embodiments the polymer particles may include biodegradable pH-sensitive polymers, such as, for example, poly(acrylic acid), poly(methyl acrylic acid) and their copolymers and derivatives, homopolymers such as poly(amino carboxylic acid), polysaccharides such as celluloseacetatephthalate, hydroxypropylmethylcellulosephthalate, hydroxypropylmethylcellulosesuccinate, celluloseacetatetrimellitate, chitosan.

In further exemplary embodiments, it can be especially preferred to select the polymer particles from biodegradable temperature sensitive polymers, such as for example, poly(N-isopropylacrylamide-co-sodium-acrylate-co-n-N-alkylacrylamide), poly(N-methyl-N-n-propylacrylamide), poly(N-methyl-N-isopropylacrylamide), poly(N—N-propylmethacrylamide), poly(N-isopropylacrylamide), poly(N,N-diethylacrylamide), poly(N-isopropylmethacrylamide), poly(N-cyclopropylacrylamide), poly(N-ethylacrylamide), poly(N-ethylmethylacrylamide), poly(N-methyl-N-ethylacrylamide), poly(N-cyclopropylacrylamide). Other polymers suitable in this regard and having thermogel characteristics include hydroxypropylcellulose, methylcellulose, hydroxypropylmethylcellulose, ethylhydroxyethylcellulose and pluronics such as F-127, L-122, L-92, L-81, L-61; functionalized dextrane or polyamino acids, such as poly-D-amino acids or poly-L-amino acids, for example polylysine, or polymers which contain lysine or other suitable amino acids. Other useful polyamino acids can include polyglutamic acids, polyaspartic acid, copolymers of lysine and glutamine or aspartic acid, copolymers of lysine with alanine, tyrosine, phenylalanine, serine, tryptophan and/or proline.

The organic particles useful according to the invention can have the same size as defined for the metal-based particles above. In exemplary embodiments, it is however preferred that the polymer particles are generally larger in size than the metal-based particles, e.g. at least 10 times or 100 times larger than the metal-based particles. Therefore, the organic polymer particles may have an average particle size from about 100 nm to 1,000 μm, preferably below about 500 μm, such as from about 100 nm to 100 μm, or below 100 μm, such as from about 500 nm to 100 μm, or in the range from about 0.7 nm to 800 nm.

### Molding

For molding the particles into a desired shape, a suspension of the particles can be formed. In the embodiments of the present invention, the first and second particles can be wetted with a solvent or suspended in a suitable solvent to form a suspension. The solvent has to be selected such that the first and second particles are substantially insoluble in the solvent. Moldable suspensions can include, depending on the particles selected, solvents such as alcohols, ethers, hydrocarbons or water. Examples include methanol, ethanol, N-propanol, isopropanol, butoxydiglycol, butoxyethanol, butoxyisopropanol, butoxypropanol, n-butyl alcohol, t-butyl alcohol, butylene glycol, butyl octanol, diethylene glycol, dimethoxydiglycol, dimethyl ether, dipropylene glycol, ethoxydiglycol, ethoxyethanol, ethyl hexane diol, glycol, hexane diol, 1,2,6-hexane triol, hexyl alcohol, hexylene glycol, isobutoxy propanol, isopentyl diol, 3-methoxybutanol, methoxydiglycol, methoxyethanol, methoxyisopropanol, methoxymethylbutanol, methoxy PEG-10, methylal, methyl hexyl ether, methyl propane diol, neopentyl glycol, PEG-4, PEG-6, PEG-7, PEG-8, PEG-9, PEG-6-methyl ether, pentylene glycol, PPG-7, PPG-2-buteth-3, PPG-2 butyl ether, PPG-3 butyl ether, PPG-2 methyl ether, PPG-3 methyl ether, PPG-2 propyl ether, propane diol, propylene glycol, propylene glycol butyl ether, propylene glycol propyl ether, tetrahydrofurane, trimethyl hexanol, phenol, benzene, toluene, xylene; as well as water, if necessary in mixture with dispersants, surfactants or other additives and mixtures of the above-named substances. In some embodiments it is suitable to use liquid nitrogen or carbon dioxide as a solvent.

The moldable suspension can have at minimum 50% by weight solids content of the metal-based particles such as about 60 to 80 wt.-%, and not more than 40 wt.-% of the solids content of the polymer particles. The solvent content in the suspension typically does not exceed 50 wt.-% of the moldable composition, such as 30 wt.-% or less than 10 wt.-%. The suspension can be viscous, such as paste-like. Typical viscosities (at 20° C.) of the moldable suspension may be above about 10³ mPa·s, e.g. at about 10³ to 10¹⁰ mPa·s, such as about 10³ to 10⁶ mPa·s, or at about 10⁴ to 10⁵ mPa·s.

If solvents are used, the solvent is typically removed during molding or after molding, for example with the use of heat, such as in a drying step, or by vacuum or at best low pressure (i.e. below normal pressure) to evaporate the solvent.

Preparation of the Suspension can be Carried Out Applying Conventional Processes to obtain substantially homogeneous suspensions. In some embodiments it can be preferred not to use any solvent, but to mix the particles based on dry methods and to mold the implant from a substantially dry powder mixture.

A variety of conventional molding techniques can be used in the embodiments of the present invention for compressing the particles and molding the implant. Such molding techniques can include, for example, injection molding, compression molding, compacting, dry pressing, cold isostatic pressing, hot pressing, uniaxial or biaxial pressing, extrusion molding, gel casting, slip casting and tape casting.

In some further exemplary embodiments, the molding process can be based on micromolding, for example for producing filigrane stent structures, screws or plates.

The suspension may preferably be compacted by appropriate means. In exemplary embodiments, the suspension is molded by injection molding.

One suitable compacting device that achieves sufficiently uniform compacting forces is a floating mold die press. The compaction pressure generally determines the density of the molded implant. If the compaction pressure is too low, the implant can have a lower than desired density and not attain the desired net shape. If the compaction pressure is too high, the molded implant can delaminate and result in a material that is defective for the intended use. The compaction pressure to obtain the implants of the embodiments of the present invention can be in the preferred ranges of from about 1,000 psi (6.89 MPa) to 20,000 psi (138 MPa), such as from about 5,000 psi to 15,000 psi, or about 10,000 psi (68.9 MPa).

The compaction time can be readily determined by the operator depending on the compaction pressure selected. Compaction time, for example, can be in the range of from about 60 seconds to 10 seconds for compaction pressures in the range of from 10,000 psi to 15,000 psi, respectively, and about 30 seconds for a compaction pressure of 12,000 psi. To produce a net shape implant according to the invention, the compacting can be carried out for a time sufficient to compact the precursor to form a molded implant having a predetermined density, for example, from about 1.0 g/cc to 15.5 g/cc. The compaction pressure and time selected by the operator can be dependent on the size of the finished part. Generally, as the part size increases, compaction pressure and/or compaction time increase. With the use of appropriate compaction techniques as herein described, the use of binders is typically not necessary to firmly adhere the particles together to form a mechanically stable product.

Another aspect of the exemplary embodiment includes the desirability for mechanical stability of the final implant. For example, for stents it is desirable to have a higher density of the particles and a more compact implant body to allow sufficient elastomechanical stability for crimping on balloon catheters and subsequent expansion during the intended use.

The molds can be selected as desired, suitable for the specific design of any implant. The implantable medical devices chosen are not limited to any particular implant type so that, for example, however not exclusively, the implant producible by the embodiments of the method of the present invention can include vessel endoprostheses, intraluminal endoprotheses, stents, coronary stents, peripheral stents, pacemakers or parts thereof, surgical and dental and orthopedic implants for temporary purposes such as joint socket inserts, surgical screws, plates, nails, implantable orthopedic supporting aids, surgical and orthopedic implants such as bones or joint prostheses, for example artificial hip or knee joints bone and body vertebra means, artificial hearts or parts thereof, artificial heart valves, cardiac pacemakers housings, electrodes, subcutaneous and/or intramuscular implants, active substance repositories or microchips or the like, also injection needles, tubes or endoscope parts or seed implants.

Without wishing to be bound to any particular theory, it is believed that using nanosized metal-based particles in compacting methods such as injection molding or extrusion molding, the molded implant can be mechanically stable without any sintering, and typically also without the addition of binders.

### Pore Design

Without referring to a specific theory, it was found that the shape and the size of the biodegradable polymer particles can result in a reproducible and rationally designable structure of the implant after degradation of the polymer particles in-vivo. For example, using fibrous polymer particles can result in forming of fibrous cavities within the implants. Using spherical particles can result in spherical cavities, whereby mixing both entities of particle types results in both formation of fibrous and spherical cavities, e.g. open porous networks.

The design of pores, pore sizes, shapes and pore volume, depends on the implant and its intended use as well as implant function. The skilled person can readily determine the amount of degradable polymer particles required to obtain a specific volume of pores left in the implant after in-vivo degradation of the polymer. Pore volumes can be increased either by using larger sized polymer particles or increasing the total amount of smaller sized polymer particles. Depending on the intended use and functional requirements in some embodiments, it may also be preferable to adjust the size of the metal-based particles in order to obtain a suitable grain size of the implant and to increase the structural integrity. The selection of the size of polymer particles can also determine the resulting size of the pores within the implant. For the polymer particles, spherical particles may be selected with a size from about 2 nm up to about 5000 μm, such as from about 10 nm up to 1000 nm or from about 100 nm up to 800 nm. In some embodiments, a structure of hierarchical porosities may be obtained by combining different sizes or shapes of polymer particles. In some embodiments, fibrous polymer particles may be used, e.g. having a thickness of about 1 nm to 5,000 μm, such as from about 20 nm to 1,000 nm, or from about 50 nm to 600 μm. The length of fibrous particles can be at about 100 nm to 10.000 μm, such as from about 100 nm to 1000 μm or from about 200 nm to 1000 nm. In some exemplary embodiments, spherical and fibrous polymer particles may be combined.

A person skilled in the art can easily calculate the ratio of both particle types based on the densities of the metal-based particles and polymer particles. To increase the mechanical stability and structural integrity of the implant, the ratio of the particle sizes of both particle types may be adjusted. In some embodiments a size ratio of metal-based particles versus polymer particles may be at about 1:1, or about 2:1, or about 5:1. In other embodiments, it can be more appropriate to use the particles in a ratio of about 1:2, or from about 1:5 or 1:20, or 1:30. Any other ratio may be suitable according to the invention, depending of the final implant and the desired shape, function and mechanical properties.

### Functional Modification

Functional modification can be carried out by adding an active ingredient to the polymer particles embedded in the implant structure. This may be done before or after molding. In certain exemplary embodiments, functional modification can involve coating the produced implant partially or completely with an active ingredient, such as therapeutically active agents, diagnostic agents or absorptive agents. In further exemplary embodiments, the therapeutically active, diagnostic or absorptive agents are part of the metal-based particles and remain a part of the implant body.

Therapeutically active agents suitable for being incorporated into the polymer particles or for being coated on at least a part of the implant according to the present invention are preferably therapeutically active agents which are capable of providing direct or indirect therapeutic, physiologic and/or pharmacologic effect in a human or animal organism. In an alternative embodiment, the active ingredient may also be a compound for agricultural purposes, for example a fertilizer, pesticide, microbicide, herbicide, algicide, etc.

The therapeutically active agent may include a drug, pro-drug, a targeting group or a drug comprising a targeting group.

The active ingredients may be in crystalline, polymorphous or amorphous form or any combination thereof in order to be used in embodiment of the present invention. Suitable therapeutically active agents may be selected from the group of enzyme inhibitors, hormones, cytokines, growth factors, receptor ligands, antibodies, antigens, ion binding agents such as crown ethers and chelating compounds, substantial complementary nucleic acids, nucleic acid binding proteins including transcription factors, toxines etc. Examples of such active agents are, for example, cytokines such as erythropoietine (EPO), thrombopoietine (TPO), interleukines (including IL-1 to IL-17), insulin, insulin-like as growth factors (including IGF-1 and IGF-2), epidermal growth factor (EGF), transforming growth factors (including TGF-alpha and TGF-beta), human growth hormone, transferring, low density lipoproteins, high density lipoproteins, leptine, VEGF, PDGF, ciliary neurotrophic factor, prolactine, adrenocorticotropic hormone (ACTH), calcitonin, human chorionic gonadotropin, cortisol, estradiol, follicle stimulating hormone (FSH), thyroid-stimulating hormone (TSH), leutinizing hormone (LH), progesterone, testosterone, toxines including ricine and further active agents such as those included in Physician's Desk Reference, 58th Edition, Medical Economics Data Production Company, Montvale, N.J., 2004 and the Merck Index, 13th Edition (particularly pages Ther-1 to Ther-29).

In one exemplary embodiment, the therapeutically active agent can be selected from the group of drugs for the therapy of oncological diseases and cellular or tissue alterations. Suitable therapeutic agents include, e.g., antineoplastic agents, including alkylating agents such as alkyl sulfonates, e.g., busulfan, improsulfan, piposulfane, aziridines such as benzodepa, carboquone, meturedepa, uredepa; ethyleneimine and methylmelamines such as altretamine, triethylene melamine, triethylene phosphoramide, triethylene thiophosphoramide, trimethylolmelamine; so-called nitrogen mustards such as chlorambucil, chlomaphazine, cyclophosphamide, estramustine, ifosfamide, mechlorethamine, mechlorethaminoxide hydrochloride, melphalan, novembichin, phenesterine, prednimustine, trofosfamide, uracil mustard; nitroso urea-compounds such as carmustine, chlorozotocin, fotenmustine, lomustine, nimustine, ranimustine; dacarbazine, mannomustine, mitobranitol, mitolactol; pipobroman; doxorubicin and cis-platinum and its derivatives, etc., and further including combinations and/or derivatives of any of the foregoing.

In a further exemplary embodiment, the therapeutically active agent is selected from the group of anti-viral and anti-bacterial agents such as aclacinomycin, actinomycin, anthramycin, azaserine, bleomycin, cuctinomycin, carubicin, carzinophilin, chromomycines, ductinomycin, daunorubicin, 6-diazo-5-oxn-1-norieucin, doxorubicin, epirubicin, mitomycins, mycophenolsäure, mogalumycin, olivomycin, peplomycin, plicamycin, porfiromycin, puromycin, streptonigrin, streptozocin, tubercidin, ubenimex, zinostatin, zorubicin, aminoglycosides or polyenes or macrolid-antibiotics, etc., combinations and/or derivatives of any of the foregoing.

In a further exemplary embodiment, the therapeutically active agent may include a radio-sensitizer drug.

In a further exemplary embodiment, the therapeutically active agent may include a steroidal or non-steroidal anti-inflammatory drug.

In a further exemplary embodiment, the therapeutically active agent is preferably selected from agents referring to angiogenesis, such as e.g. endostatin, angiostatin, interferones, platelet factor 4 (PF4), thrombospondin, transforming growth factor beta, tissue inhibitors of the metalloproteinases-1, -2 and -3 (TIMP-1, -2 and -3), TNP-470, marimastat, neovastat, BMS-275291, COL-3, AG3340, thalidomide, squalamine, combrestastatin, SU5416, SU6668, IFN-[alpha], EMD121974, CAI, IL-12 and IM862 etc., and further including combinations and/or derivatives of any of the foregoing.

In a further exemplary embodiment, the therapeutically-active agent can be selected from the group of nucleic acids, wherein the term nucleic acids also includes oligonucleotides, wherein at least two nucleotides are covalently linked to each other, for example in order to provide gene therapeutic or antisense effects. Nucleic acids preferably comprise phosphodiester bonds, which also comprise those which are analogues having different backbones. Analogues may also contain backbones such as, for example, phosphoramide (Beaucage et al., Tetrahedron 49(10):1925 (1993) and the references cited therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:1419 (1986)); phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989)), O-methylphosphoroamidit-compounds (see Eckstein, Oligonucleotides and Analogues: A Practical Approach, Oxford University Press), and peptide-nucleic acid-backbones and their compounds (see Egholm, J. Am. Chem. Soc. 114:1895 (1992); Meier et al., Chem. Int. Ed. Engl: 31:1008 (1992); Nielsen, Nature, 365:566 (1993); Carlsson et al., Nature 380:207 (1996), wherein the disclosure of these references are incorporated by reference in their entirety. Further analogues are those having ionic backbones, see Denpcy et al., Proc. Natl. Acad. Sci. USA 92:6097 (1995), or non-ionic backbones, see U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996), and non-ribose-backbones, including those which are described in U.S. Pat. Nos. 5,235,033 and 5,034,506, and in chapters 6 and 7 of ASC Symposium Series 580, “Carbohydrate Modifications in Antisense Research”, Ed. Y. S. Sanghui and P. Dan Cook). The nucleic acids having one or more carbocylic sugars are also suitable as nucleic acids for use in the present invention, see Jenkins et al., Chemical Society Review (1995), pages 169 to 176 as well as others which are described in Rawls, C & E News, 2 Jun. 1997, page 36. The references disclose herewith are incorporated by reference in their entirety. Besides the selection of the nucleic acids and nucleic acid analogues known in the prior art, also a mixture of naturally occurring nucleic acids and nucleic acid analogues or mixtures of nucleic acid analogues may be used.

In a further embodiment, the therapeutically active agent can selected from the group of metal ion complexes, such as described in PCT US95/16377, PCT US95/16377, PCT US96/19900, PCT US96/15527, wherein such agents reduce or inactivate the bioactivity of their target molecules, preferably proteins such as enzymes.

Therapeutically active agents may also include anti-migratory, anti-proliferative or immune-supressive, anti-inflammatory or re-endotheliating agents such as, e.g., everolimus, tacrolimus, sirolimus, mycofenolate-mofetil, rapamycin, paclitaxel, actinomycine D, angiopeptin, batimastate, estradiol, VEGF, statines and others, their derivatives and analogues.

Active agents or combinations of active agents may further be selected from heparin, synthetic heparin analogues (e.g., fondaparinux), hirudin, antithrombin III, drotrecogin alpha; fibrinolytics such as alteplase, plasmin, lysokinases, factor XIIa, prourokinase, urokinase, anistreplase, streptokinase; platelet aggregation inhibitors such as acetylsalicylic acid [aspirin], ticlopidine, clopidogrel, abciximab, dextrans; corticosteroids such as alclometasone, amcinonide, augmented betamethasone, beclomethasone, betamethasone, budesonide, cortisone, clobetasol, clocortolone, desonide, desoximetasone, dexamethasone, fluocinolone, fluocinonide, flurandrenolide, flunisolide, fluticasone, halcinonide, halobetasol, hydrocortisone, methylprednisolone, mometasone, prednicarbate, prednisone, prednisolone, triamcinolone; so-called non-steroidal anti-inflammatory drugs (NSAIDs) such as diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, piroxicam, salsalate, sulindac, tolmetin, celecoxib, rofecoxib; cytostatics such as alkaloids and podophyllum toxins such as vinblastine, vincristine; alkylating agents such as nitrosoureas, nitrogen lost analogues; cytotoxic antibiotics such as daunorubicin, doxorubicin and other anthracyclines and related substances, bleomycin, mitomycin; antimetabolites such as folic acid analogues, purine analogues or pyrimidine analogues; paclitaxel, docetaxel, sirolimus; platinum compounds such as carboplatin, cisplatin or oxaliplatin; amsacrin, irinotecan, imatinib, topotecan, interferon-alpha 2a, interferon-alpha 2b, hydroxycarbamide, miltefosine, pentostatin, porfimer, aldesleukin, bexaroten, tretinoin; antiandrogens and antiestrogens; antiarrhythmics, in particular class I antiarrhythmic such as antiarrhythmics of the quinidine type, quinidine, dysopyramide, ajmaline, prajmalium bitartrate, detajmium bitartrate; antiarrhythmics of the lidocaine type, e.g., lidocaine, mexiletin, phenyloin, tocainid; class Ic antiarrhythmics, e.g., propafenon, flecainid(acetate); class II antiarrhythmics beta-receptor blockers such as metoprolol, esmolol, propranolol, metoprolol, atenolol, oxprenolol; class III antiarrhythmics such as amiodarone, sotalol; class IV antiarrhythmics such as diltiazem, verapamil, gallopamil; other antiarrhythmics such as adenosine, orciprenaline, ipratropium bromide; agents for stimulating angiogenesis in the myocardium such as vascular endothelial growth factor (VEGF), basic fibroblast growth factor (bFGF), non-viral DNA, viral DNA, endothelial growth factors: FGF-1, FGF-2, VEGF, TGF; antibiotics, monoclonal antibodies, anticalins; stem cells, endothelial progenitor cells (EPC); digitalis glycosides, such as acetyl digoxin/metildigoxin, digitoxin, digoxin; cardiac glycosides such as ouabain, proscillaridin; antihypertensives such as CNS active antiadrenergic substances, e.g., methyldopa, imidazoline receptor agonists; calcium channel blockers of the dihydropyridine type such as nifedipine, nitrendipine; ACE inhibitors: quinaprilate, cilazapril, moexipril, trandolapril, spirapril, imidapril, trandolapril; angiotensin II antagonists: candesartancilexetil, valsartan, telmisartan, olmesartanmedoxomil, eprosartan; peripherally active alpha-receptor blockers such as prazosin, urapidil, doxazosin, bunazosin, terazosin, indoramin; vasodilatators such as dihydralazine, diisopropylamine dichloracetate, minoxidil, nitroprusside sodium; other antihypertensives such as indapamide, co-dergocrine mesylate, dihydroergotoxin methanessulfonate, cicletanin, bosentan, fludrocortisone; phosphodiesterase inhibitors such as milrinon, enoximon and antihypotensives such as in particular adrenergic and dopaminergic substances such as dobutamine, epinephrine, etilefrine, norfenefrine, norepinephrine, oxilofrine, dopamine, midodrine, pholedrine, ameziniummetil; and partial adrenoceptor agonists such as dihydroergotamine; fibronectin, polylysine, ethylene vinyl acetate, inflammatory cytokines such as: TGF, PDGF, VEGF, bFGF, TNF, NGF, GM-CSF, IGF-a, IL-1, IL 8, IL-6, growth hormone; as well as adhesive substances such as cyanoacrylates, beryllium, silica; and growth factors such as erythropoetin, hormones such as corticotropins, gonadotropins, somatropins, thyrotrophins, desmopressin, terlipressin, pxytocin, cetrorelix, corticorelin, leuprorelin, triptorelin, gonadorelin, ganirelix, buserelin, nafarelin, goserelin, as well as regulatory peptides such as somatostatin, octreotid; bone and cartilage stimulating peptides, bone morphogenetic proteins (BMPs), in particular recombinant BMPs, such as recombinant human BMP-2 (rhBMP-2), bisphosphonate (e.g., risedronate, pamidronate, ibandronate, zoledronic acid, clodronic acid, etidronic acid, alendronic acid, tiludronic acid), fluorides such as disodium fluorophosphate, sodium fluoride; calcitonin, dihydrotachystyrol; growth factors and cytokines such as epidermal growth factor (EGF), platelet-derived growth factor (PDGF), fibroblast growth factors (FGFs), transforming growth factors-b (TGFs-b), transforming growth factor-a (TGF-a), erythropoietin (EPO), insulin-like as growth factor-I (IGF-I), insulin-like as growth factor-II (IGF-II), interleukin-1 (IL-1), interleukin-2 (IL-2), interleukin-6 (IL-6), interleukin-8 (IL-8), tumor necrosis factor-a (TNF-a), tumor necrosis factor-b (TNF-b), interferon-g (INF-g), colony stimulating factors (CSFs); monocyte chemotactic protein, fibroblast stimulating factor 1, histamine, fibrin or fibrinogen, endothelin-1, angiotensin II, collagens, bromocriptine, methysergide, methotrexate, carbon tetrachloride, thioacetamide and ethanol; as well as silver (ions), titanium dioxide, antibiotics and anti-infective drugs such as, in particular, β-lactam antibiotics, e.g., β-lactamase-sensitive penicillins such as benzyl penicillins (penicillin G), phenoxymethylpenicillin (penicillin V); β-lactamase-resistent penicillins such as aminopenicillins, e.g., amoxicillin, ampicillin, bacampicillin; acylaminopenicillins such as mezlocillin, piperacillin; carboxypenicillins, cephalosporins such as cefazoline, cefuroxim, cefoxitin, cefotiam, cefaclor, cefadroxil, cefalexin, loracarbef, cefixim, cefuroximaxetil, ceftibuten, cefpodoximproxetil, cefpodoximproxetil; aztreonam, ertapenem, meropenem; β-lactamase inhibitors such as sulbactam, sultamicillintosylate; tetracyclines such as doxycycline, minocycline, tetracycline, chlorotetracycline, oxytetracycline; aminoglycosides such as gentamicin, neomycin, streptomycin, tobramycin, amikacin, netilmicin, paromomycin, framycetin, spectinomycin; macrolide antibiotics such as azithromycin, clarithromycin, erythromycin, roxithromycin, spiramycin, josamycin; lincosamides such as clindamycin, lincomycin; gyrase inhibitors such as fluoroquinolones, e.g., ciprofloxacin, ofloxacin, moxifloxacin, norfloxacin, gatifloxacin, enoxacin, fleroxacin, levofloxacin; quinolones such as pipemidic acid; sulfonamides, trimethoprim, sulfadiazine, sulfalene; glycopeptide antibiotics such as vancomycin, teicoplanin; polypeptide antibiotics such as polymyxins, e.g., colistin, polymyxin-b, nitroimidazole derivates, e.g., metronidazole, tinidazole; aminoquinolones such as chloroquin, mefloquin, hydroxychloroquin; biguanids such as proguanil; quinine alkaloids and diaminopyrimidines such as pyrimethamine; amphenicols such as chloramphenicol; rifabutin, dapson, fusidic acid, fosfomycin, nifuratel, telithromycin, fusafungin, fosfomycin, pentamidine diisethionate, rifampicin, taurolidin, atovaquon, linezolid; virus static such as aciclovir, ganciclovir, famciclovir, foscarnet, inosine-(dimepranol-4-acetamidobenzoate), valganciclovir, valaciclovir, cidofovir, brivudin; antiretroviral active ingredients (nucleoside analogue reverse-transcriptase inhibitors and derivatives) such as lamivudine, zalcitabine, didanosine, zidovudin, tenofovir, stavudin, abacavir; non-nucleoside analogue reverse-transcriptase inhibitors: amprenavir, indinavir, saquinavir, lopinavir, ritonavir, nelfinavir; amantadine, ribavirine, zanamivir, oseltamivir or lamivudine, as well as any combinations and mixtures thereof.

In an alternative embodiment of the present invention, the active agents can be encapsulated in polymers, vesicles, liposomes or micelles.

Suitable diagnostically active agents can be e.g. signal generating agents or materials which may be used as markers. Such signal generating agents are materials which in physical, chemical and/or biological measurement and verification methods lead to detectable signals, for example, in image-producing methods. It is not important for the present invention whether the signal processing is carried out exclusively for diagnostic or therapeutic purposes. Typical imaging methods are, for example, radiographic methods, which are based on ionizing radiation, for example, conventional X-ray methods and X-ray based split image methods such as computer tomography, neutron transmission tomography, radiofrequency magnetization such as magnetic resonance tomography, further by radionuclide-based methods such as scintigraphy, Single Photon Emission Computed Tomography (SPECT), Positron Emission Computed Tomography (PET), ultrasound-based methods or fluoroscopic methods or luminescence or fluorescence-based methods such as Intravasal Fluorescence Spectroscopy, Raman spectroscopy, Fluorescence Emission Spectroscopy, Electrical Impedance Spectroscopy, colorimetry, optical coherence tomography, etc, further Electron Spin Resonance (ESR), Radio Frequency (RF) and Microwave Laser and similar methods.

Signal generating agents preferably can be metal-based from the group of metals, metal oxides, metal carbides, metal nitrides, metal oxynitrides, metal carbonitrides, metal oxycarbides, metal oxynitrides, metal oxycarbonitrides, metal hydrides, metal alkoxides, metal halides, inorganic or organic metal salts, metal polymers, metallocenes, and other organometallic compounds.

Preferred metal-based agents are especially nanomorphous nanoparticles from metals, metal oxide semiconductors as defined above as the metal-based particles, or mixtures thereof. In this regard, it may be preferred to select at least a part of the metal-based particles from those materials capable of functioning as signal generating agents, for example to mark the implant for better visibility and localization in the body after implantation.

Further, signal producing metal-based agents can be selected from salts or metal ions, which preferably have paramagnetic properties, for example lead (II), bismuth (II), bismuth (III), chromium (III), manganese (II), manganese (III), iron (II), iron (III), cobalt (II), nickel (II), copper (II), praseodymium (III), neodymium (III), samarium (III), or ytterbium (III), holmium (III) or erbium (III) and the like. For especially pronounced magnetic moments, gadolinium (III), terbium (III), dysprosium (III), holmium (III) and erbium (III) are particularly preferred. Further one can select from radioisotopes. Examples of a few applicable radioisotopes include H 3, Be 10, O 15, Ca 49, Fe 60, In 111, Pb 210, Ra 220, Ra 224 and the like. Typically, such ions are present as chelates or complexes, wherein for example as chelating agents or ligands for lanthanides and paramagnetic ions compounds such as diethylenetriamine pentaacetic acid (“DTPA”), ethylenediamine tetra acetic acid (“EDTA”), or tetraazacyclododecane-N,N′,N″,N′″-tetra acetic acid (“DOTA”) are used. Other typical organic complexing agents are, for example, published in Alexander, Chem. Rev. 95:273-342 (1995) and Jackels, Pharm. Med. Imag, Section III, Chap. 20, p 645 (1990). Other usable chelating agents may be found in U.S. Pat. Nos. 5,155,215; 5,087,440; 5,219,553; 5,188,816; 4,885,363; 5,358,704; 5,262,532, and Meyer et al., Invest. Radiol. 25: S53 (1990), further U.S. Pat. Nos. 5,188,816, 5,358,704, 4,885,363, and 5,219,553. These patents and the cited portions of the non-patent publications are hereby incorporated by reference in their entireties. Also, salts and chelates from the lanthanide group with the atomic numbers 57-83 or the transition metals with the atomic numbers 21-29, or 42 or 44 may be incorporated into the implants of exemplary embodiments of the present invention.

Also suitable can be paramagnetic perfluoroalkyl-containing compounds, which, for example, are described in German laid-open patents DE 196 03 033, DE 197 29 013 and in WO 97/26017. further diamagnetic perfluoroalkyl containing substances of the general formula:

R<PF>-L<II>-G<III>,

wherein R<PF> represents a perfluoroalkyl group with 4 to 30 carbon atoms, L<II> stands for a linker and G<III> for a hydrophilic group. The linker L is a direct bond, an —SO2-group or a straight or branched carbon chain with up to 20 carbon atoms which can be substituted with one or more —OH, —COO<->, —SO3-groups and/or if necessary one or more —O—, —S—, —CO—, —CONH—, —NHCO—, —CONR—, —NRCO—, —SO2-, —PO4-, —NH—, —NR-groups, an aryl ring or contain a piperazine, wherein R stands for a C1 to C20 alkyl group, which again can contain and/or have one or a plurality of O atoms and/or be substituted with —COO<-> or SO3-groups.

The hydrophilic group G<III> can be selected from a mono or disaccharide, one or a plurality of —COO<-> or —SO3<->-groups, a dicarboxylic acid, an isophthalic acid, a picolinic acid, a benzenesulfonic acid, a tetrahydropyranedicarboxylic acid, a 2,6-pyridinedicarboxylic acid, a quaternary ammonium ion, an aminopolycarboxcylic acid, an aminodipolyethyleneglycol sulfonic acid, an aminopolyethyleneglycol group, an SO2-(CH2)₂—OH-group, a polyhydroxyalkyl chain with at least two hydroxyl groups or one or a plurality of polyethylene glycol chains having at least two glycol units, wherein the polyethylene glycol chains are terminated by an —OH or —OCH3-group, or similar linkages.

In an exemplary embodiment, paramagnetic metals in the form of metal complexes with phthalocyanines may be used to functionalize the implant, such as described in Phthalocyanine Properties and Applications, Vol. 14, C. C. Leznoff and A. B. P. Lever, VCH Ed. Examples are octa(1,4,7,10-tetraoxaundecyl)Gd-phthalocyanine, octa(1,4,7,10-tetraoxaundecyl)Gd-phthalocyanine, octa(1,4,7,10-tetraoxaundecyl)Mn-phthalocyanine, octa(1,4,7,10-tetraoxaundecyl)Mn-phthalocyanine, as described in U.S. 2004/214810, super-paramagnetic, ferromagnetic or ferrimagnetic signal generating agents may also be used. For example, among magnetic metals, alloys are preferred, among ferrites such as gamma iron oxide, magnetites or cobalt-, nickel- or manganese-ferrites, corresponding agents are preferably selected, especially particles, as described in WO83/03920, WO83/01738, WO85/02772 and WO89/03675, in U.S. Pat. No. 4,452,773, U.S. Pat. No. 4,675,173, in WO88/00060 as well as U.S. Pat. No. 4,770,183, in WO90/01295 and in WO90/01899.

Further, magnetic, paramagnetic, diamagnetic or super paramagnetic metal oxide crystals having diameters of less than 4000 Angstroms are especially preferred as degradable non-organic diagnostic agents. Suitable metal oxides can be selected from iron oxide, cobalt oxides, iridium oxides or the like, which provide suitable signal-producing properties and which have especially biocompatible properties or are biodegradable. Crystalline agents of this group having diameters smaller than 500 Angstroms may also be used. These crystals can be associated covalently or non-covalently with macromolecular species. Further, zeolite-containing paramagnets and gadolinium-containing nanoparticles can be selected from polyoxometallates, preferably of the lanthanides, (e.g., K9GdW10O36).

To optimize the image-producing properties the average particle size of the magnetic signal producing agents may be limited to 5 μm at maximum, such as from about 2 nm up to 1 μm, e.g. from about 5 nm to 200 nm. The super paramagnetic signal producing agents can be chosen, for example, from the group of so-called SPIOs (super paramagnetic iron oxides) with a particle size larger than 50 nm or from the group of the USPIOs (ultra small super paramagnetic iron oxides) with particle sizes smaller than 50 nm.

Signal generating agents for imparting further functionality to the implants of embodiments of the present invention can further be selected from endohedral fullerenes, as disclosed, for example, in U.S. Pat. No. 5,688,486 or WO 93/15768, or from fullerene derivatives and their metal complexes such as fullerene species, which comprise carbon clusters having 60, 70, 76, 78, 82, 84, 90, 96 or more carbon atoms. An overview of such species can be gathered from European patent application 1331226A2. Metal fullerenes or endohedral carbon nanoparticles with arbitrary metal-based components can also be selected. Such endohedral fullerenes or endometallo fullerenes may contain, for example, rare earths such as cerium, neodymium, samarium, europium, gadolinium, terbium, dysprosium or holmium. The choice of nanomorphous carbon species is not limited to fullerenes, other nanomorphous carbon species such as nanotubes, onions, etc., may also be applicable.

In another exemplary embodiment, fullerene species may be selected from non-endohedral or endohedral forms which contain halogenated, preferably iodated groups, as disclosed in U.S. Pat. No. 6,660,248.

Generally, mixtures of such signal generating agents of different specifications can also be used, depending on the desired properties of the signal generating material properties. The signal-producing agents used can have a size of 0.5 nm to 1,000 nm, preferably 0.5 nm to 900 nm, especially preferred from 0.7 to 100 nm, and they may partly replace the metal-based particles. Nanoparticles are generally easily modifiable based on their large surface to volume ratios. The nanoparticles can be, for example, modified non-covalently by means of hydrophobic ligands, for example with trioctylphosphine, or be covalently modified. Examples of covalent ligands include thiol fatty acids, amino fatty acids, fatty acid alcohols, fatty acids, fatty acid ester groups or mixtures thereof, for example oleic acid and oleylamine.

In exemplary embodiments of the invention, the signal-producing agents can be encapsulated in micelles or liposomes with the use of amphiphilic components, or may be encapsulated in polymeric shells, wherein the micelles/liposomes can have a diameter of 2 nm to 800 nm, preferably from 5 to 200 nm, especially preferred from 10 to 25 nm. The micelles/liposomes may be added to the suspension before molding, to be incorporated into the implant. The size of the micelles/liposomes is, without committing to a specific theory, dependant on the number of hydrophobic and hydrophilic groups, the molecular weight of the nanoparticles and the aggregation number. In aqueous solutions, the use of branched or unbranched amphiphilic substances is especially preferred in order to achieve the encapsulation of signal generating agents in liposomes/micelles. The hydrophobic nucleus of the micelles hereby contains in an exemplary embodiment a multiplicity of hydrophobic groups, preferably between 1 and 200, especially preferred between 1 and 100 and mostly preferred between 1 and 30 according to the desired setting of the micelle size.

Hydrophobic groups consist preferably of hydrocarbon groups or residues or silicon-containing residues, for example polysiloxane chains. Furthermore, they can preferably be selected from hydrocarbon-based monomers, oligomers and polymers, or from lipids or phospholipids or comprise combinations hereof, especially glyceryl esters such as phosphatidyl ethanolamine, phosphatidyl choline, or polyglycolides, polylactides, polymethacrylate, polyvinylbutylether, polystyrene, polycyclopentadienylmethylnorbornene, polyethylenepropylene, polyethylene, polyisobutylene, polysiloxane. Further for encapsulation in micelles hydrophilic polymers are also selected, especially preferred polystyrenesulfonic acid, poly-N-alkylvinylpyridiniumhalides, poly(meth)acrylic acid, polyamino acids, poly-N-vinylpyrrolidone, polyhydroxyethylmethacrylate, polyvinyl ether, polyethylene glycol, polypropylene oxide, polysaccharides such as agarose, dextrane, starches, cellulose, amylose, amylopectin, or polyethylene glycol or polyethylene imine of any desired molecular weight, depending on the desired micelles property. Further, mixtures of hydrophobic or hydrophilic polymers can be used or such lipid-polymer compositions employed. In a further particular embodiment, the polymers can be used as conjugated block polymers, wherein hydrophobic and also hydrophilic polymers or any desired mixtures thereof can be selected as 2-, 3- or multi-block copolymers.

Such signal generating agents encapsulated in micelles can, moreover, be functionalized, while linker (groups) are attached at any desired position, preferably amino-, thiol, carboxyl-, hydroxyl-, succinimidyl, maleimidyl, biotin, aldehyde- or nitrilotriacetate groups, to which any desired corresponding chemically covalent or non-covalent other molecules or compositions can be bound according to the prior art. Here, especially biological molecules such as proteins, peptides, amino acids, polypeptides, lipoproteins, glycosaminoglycanes, DNA, RNA or similar bio molecules are preferred especially.

Signal generating agents may also be selected from non-metal-based signal generating agents, for example from the group of X-ray contrast agents, which can be ionic or non-ionic. Included among the ionic contrast agents are, for example, salts of 3-acetyl amino-2,4-6-triiodobenzoic acid, 3,5-diacetamido-2,4,6-triiodobenzoic acid, 2,4,6-triiodo-3,5-dipropionamido-benzoic acid, 3-acetyl amino-5-((acetyl amino)methyl)-2,4,6-triiodobenzoic acid, 3-acetyl amino-5-(acetyl methyl amino)-2,4,6-triiodobenzoic acid, 5-acetamido-2,4,6-triiodo-N-((methylcarbamoyl)methyl)-isophthalamic acid, 5-(2-methoxyacetamido)-2,4,6-triiodo-N-[2-hydroxy-1-(methylcarbamoyl)-ethoxy 1]-isophthalamic acid, 5-acetamido-2,4,6-triiodo-N-methylisophthalamic acid, 5-acetamido-2,4,6-triiodo-N-(2-hydroxyethyl)-isophthalamic acid 2-[[2,4,6-triiodo-3-[(1-oxobutyl)-amino]phenyl]methyl]-butanoic acid, beta-(3-amino-2,4,6-triiodophenyl)-alpha-ethyl-propanoic acid, 3-ethyl-3-hydroxy-2,4,6-triiodophenyl-propanoic acid, 3-[[(dimethylamino)-methyl]amino]-2,4,6-triiodophenyl-propanoic acid (see Chem. Ber. 93: 2347 (1960)), alpha-ethyl-(2,4,6-triiodo-3-(2-oxo-1-pyrrolidinyl)-phenyl)-propanoic acid, 2-[2-[3-(acetyl amino)-2,4,6-triiodophenoxy]ethoxymethyl]butanoic acid, N-(3-amino-2,4,6-triiodobenzoyl)-N-phenyl-.beta.-aminopropanoic acid, 3-acetyl-[(3-amino-2,4,6-triiodophenyl)amino]-2-methylpropanoic acid, 5-[(3-amino-2,4,6-triiodophenyl)methyl amino]-5-oxypentanoic acid, 4-[ethyl-[2,4,6-triiodo-3-(methyl amino)-phenyl]amino]-4-oxo-butanoic acid, 3,3′-oxy-bis[2,1-ethanediyloxy-(1-oxo-2,1-ethanediyl)imino]bis-2,4,6-triiodobenzoic acid, 4,7,10,13-tetraoxahexadecane-1,16-dioyl-bis(3-carboxy-2,4,6-triiodoanilide), 5,5′-(azelaoyldiimino)-bis[2,4,6-triiodo-3-(acetyl amino)methyl-benzoic acid], 5,5′-(apidoldiimino)bis(2,4,6-triiodo-N-methyl-isophthalamic acid), 5,5′-(sebacoyl-diimino)-bis(2,4,6-triiodo-N-methylisophthalamic acid), 5,5-[N,N-diacetyl-(4,9-dioxy-2,11-dihydroxy-1,12-dodecanediyl)diimino]bis(2,4,6-triiodo-N-methyl-isophthalamic acid), 5,5′5″-(nitrilo-triacetyltriimino)tris(2,4,6-triiodo-N-methyl-isophthalamic acid), 4-hydroxy-3,5-diiodo-alpha-phenylbenzenepropanoic acid, 3,5-diiodo-4-oxo-1(4H)-pyridine acetic acid, 1,4-dihydro-3,5-diiodo-1-methyl-4-oxo-2,6-pyridinedicarboxylic acid, 5-iodo-2-oxo-1(2H)-pyridine acetic acid, and N-(2-hydroxyethyl)-2,4,6-triiodo-5-[2,4,6-triiodo-3-(N-methylacetamido)-5-(methylcarbomoyl)benzamino]acetamido]-isophthalamic acid, and such as especially preferred, as well as other ionic X-ray contrast agents suggested in the literature, for example in J. Am. Pharm. Assoc., Sci. Ed. 42:721 (1953), Swiss Patent 480071, JACS 78:3210 (1956), German patent 2229360, U.S. Pat. No. 3,476,802, Arch. Pharm. (Weinheim, Germany) 306: 11 834 (1973), J. Med. Chem. 6: 24 (1963), FR-M-6777, Pharmazie 16: 389 (1961), U.S. Pat. No. 2,705,726, U.S. Pat. No. 2,895,988, Chem. Ber. 93:2347 (1960), SA-A-68/01614, Acta Radiol. 12: 882 (1972), British Patent 870321, Rec. Trav. Chim. 87: 308 (1968), East German Patent 67209, German Patent 2050217, German Patent 2405652, Farm Ed. Sci. 28: 912 (1973), Farm Ed. Sci. 28: 996 (1973), J. Med. Chem. 9: 964 (1966), Arzheim.-Forsch 14: 451 (1964), SE-A-344166, British Patent 1346796, U.S. Pat. No. 2,551,696, U.S. Pat. No. 1,993,039, Ann 494: 284 (1932), J. Pharm. Soc. (Japan) 50: 727 (1930), and U.S. Pat. No. 4,005,188.

Examples of applicable non-ionic X-ray contrast agents in accordance with an embodiment of the present invention include metrizamide as disclosed in DE-A-2031724, iopamidol as disclosed in BE-A-836355, iohexyl as disclosed in GB-A-1548594, iotrolan as disclosed in EP-A-33426, iodecimol as disclosed in EP-A-49745, iodixanol as in EP-A-108638, ioglucol as disclosed in U.S. Pat. No. 4,314,055, ioglucomide as disclosed in BE-A-846657, ioglunioe as in DE-A-2456685, iogulamide as in BE-A-882309, iomeprol as in EP-A-26281, iopentol as EP-A-105752, iopromide as in DE-A-2909439, iosarcol as in DE-A-3407473, iosimide as in DE-A-3001292, iotasul as in EP-A-22056, iovarsul as disclosed in EP-A-83964 or ioxilan in WO87/00757.

Agents based on nanoparticle signal generating agents may be selected to impart functionality to the implant, which after release into tissues and cells can be incorporated or may be enriched in intermediate cell compartments and/or have an especially long residence time in the organism.

Such particles can include water-insoluble agents, a heavy element such as iodine or barium, PH-50 as monomer, oligomer or polymer (iodinated aroyloxy ester having the empirical formula C19H23I3N₂O6, and the chemical names 6-ethoxy-6-oxohexy-3,5-bis(acetyl amino)-2,4,6-triiodobenzoate), an ester of diatrizoic acid, an iodinated aroyloxy ester, or combinations thereof. Particle sizes which can be incorporated by macrophages may be preferred. A corresponding method for this is disclosed in WO03/039601 and suitable agents are disclosed in the publications U.S. Pat. Nos. 5,322,679, 5,466,440, 5,518,187, 5,580,579, and 5,718,388. Nanoparticles which are marked with signal generating agents or such signal generating agents such as PH-50, which accumulate in intercellular spaces and can make interstitial as well as extrastitial compartments visible, can also be advantageous.

Signal generating agents may also include anionic or cationic lipids, as disclosed in U.S. Pat. No. 6,808,720, for example, anionic lipids such as phosphatidyl acid, phosphatidyl glycerol and their fatty acid esters, or amides of phosphatidyl ethanolamine, such as anandamide and methanandamide, phosphatidyl serine, phosphatidyl inositol and their fatty acid esters, cardiolipin, phosphatidyl ethylene glycol, acid lysolipids, palmitic acid, stearic acid, arachidonic acid, oleic acid, linoleic acid, linolenic acid, myristic acid, sulfolipids and sulfatides, free fatty acids, both saturated and unsaturated and their negatively charged derivatives, etc. Moreover, halogenated, in particular fluorinated anionic lipids can be preferred in exemplary embodiments. The anionic lipids preferably contain cations from the alkaline earth metals beryllium (Be<+2>), magnesium (Mg<+2>), calcium (Ca<+2>), strontium (Sr<+2>) und barium (Ba<+2>), or amphoteric ions, such as aluminium (Al<+3>), gallium (Ga<+3>), germanium (Ge<+3>), tin (Sn+<4>) or lead (Pb<+2> and Pb<+4>), or transition metals such as titanium (Ti<+3> and Ti<+4>), vanadium (V<+2> and V<+3>), chromium (Cr<+2> and Cr<+3>), manganese (Mn<+2> and Mn<+3>), iron (Fe<+2> and Fe<+3>), cobalt (Co<+2> and Co<+3>), nickel (Ni<+2> and Ni<+3>), copper (Cu<+2>), zinc (Zn<+2>), zirconium (Zr<+4>), niobium (Nb<+3>), molybdenum (Mo<+2> und Mo<+3>), cadmium (Cd<+2>), indium (In <+3>), tungsten (W<+2> and W<+4>), osmium (Os<+2>, Os<+3> and Os<+4>), iridium (Ir<+2>, Ir<+3> und Ir<+4>), mercury (Hg<+2>) or bismuth (Bi<+3>), and/or rare earths such as lanthanides, for example lanthanum (La<+3>) and gadolinium (Gd<+3>). Cations can include calcium (Ca<+2>), magnesium (Mg<+2>) and zinc (Zn<+2>) and paramagnetic cations such as manganese (Mn<+2>) or gadolinium (Gd<+3>).

Cationic lipids may include phosphatidyl ethanolamine, phospatidylcholine, Glycero-3-ethylphosphatidylcholine and their fatty acid esters, di- and tri-methylammoniumpropane, di- and tri-ethylammoniumpropane and their fatty acid esters, and also derivatives such as N-[1-(2,3-dioleoyloxy)propyl]-N,N,N-trimethylammonium chloride (“DOTMA”); furthermore, synthetic cationic lipids based on, for example, naturally occurring lipids such as dimethyldioctadecylammonium bromide, sphingolipids, sphingomyelin, lysolipids, glycolipids such as, for example, gangliosides GM1, sulfatides, glycosphingolipids, cholesterol und cholesterol esters or salts, N-succinyldioleoylphosphattidyl ethanolamine, 1,2,-dioleoyl-sn-glycerol, 1,3-dipalmitoyl-2-succinylglycerol, 1,2-dipalmitoyl-sn-3-succinylglycerol, 1-hexadecyl-2-palmitoylglycerophosphatidyl ethanolamine and palmitoyl-homocystein, and fluorinated, derivatized cationic lipids, as disclosed in U.S. Pat. No. 5,830,430. Such lipids are furthermore suitable as components of signal generating liposomes, which especially can have pH-sensitive properties as disclosed in U.S. Published Application No. 2004197392 and incorporated herein by reference in their entirety.

Other signal generating agents can be selected from agents, which are transformed into signal generating agents in organisms by means of in-vitro or in-vivo cells, cells as a component of cell cultures, of in-vitro tissues, or cells as a component of multicellular organisms, such as, for example, fungi, plants or animals, in exemplary embodiments from mammals such as mice or humans. Such agents can be made available in the form of vectors for the transfection of multicellular organisms, wherein the vectors contain recombinant nucleic acids for the coding of signal generating agents. In exemplary embodiments, this may be done with signal generating agents such as metal-binding proteins. It can be preferred to choose such vectors from the group of viruses, for example from adeno viruses, adeno virus associated viruses, herpes simplex viruses, retroviruses, alpha viruses, pox viruses, arena-viruses, vaccinia viruses, influenza viruses, polio viruses or hybrids of any of the above.

Such signal generating agents may be used in combination with delivery systems, e.g., in order to incorporate nucleic acids, which are suitable for coding for signal generating agents, into the target structure. Virus particles for the transfection of mammalian cells may be used, wherein the virus particle contains one or a plurality of coding sequence/s for one or a plurality of signal generating agents as described above. In these cases the particles can be generated from one or a plurality of the following viruses: adeno viruses, adeno virus associated viruses, herpes simplex viruses, retroviruses, alpha viruses, pox viruses, arena-viruses, vaccinia viruses, influenza viruses and polio viruses.

These signal generating agents can be made available from colloidal suspensions or emulsions, which are suitable to transfect cells, preferably mammalian cells, wherein these colloidal suspensions and emulsions contain those nucleic acids which possess one or a plurality of the coding sequence(s) for signal generating agents. Such colloidal suspensions or emulsions can include macromolecular complexes, nano capsules, microspheres, beads, micelles, oil-in-water- or water-in-oil emulsions, mixed micelles and liposomes or any desired mixture of the above.

Further, cells, cell cultures, organized cell cultures, tissues, organs of desired species and non-human organisms can be chosen which contain recombinant nucleic acids having coding sequences for signal generating agents. In exemplary embodiments, organisms can include mouse, rat, dog, monkey, pig, fruit fly, nematode worms, fish or plants or fungi. Further, cells, cell cultures, organized cell cultures, tissues, organs of desired species and non-human organisms can contain one or a plurality of vectors as described above.

Signal generating agents can be produced in vivo from proteins and made available as described above. Such agents can be directly or indirectly signal producing, while the cells produce (direct) a signal producing protein through transfection, or produce a protein which induces (indirect) the production of a signal producing protein. These signal generating agents are e.g. detectable in methods such as MRI, while the relaxation times T1, T2, or both are altered and lead to signal producing effects which can be processed sufficiently for imaging. Such proteins can include protein complexes, such as metalloprotein complexes. Direct signal producing proteins can include such metalloprotein complexes which are formed in the cells. Indirect signal producing agents can include proteins or nucleic acids, for example, which regulate the homeostasis of iron metabolism, the expression of endogenous genes for the production of signal generating agents, and/or the activity of endogenous proteins with direct signal generating properties, for example Iron Regulatory Protein (IRP), transferrin receptor (for the take-up of Fe), erythroid-5-aminobevulinate synthase (for the utilization of Fe, H-Ferritin and L-Ferritin for the purpose of Fe storage). In exemplary embodiments, both types of signal generating agents, that is direct and indirect, may be combined with each other, for example an indirect signal generating agent, which regulates the iron-homeostasis and a direct agent, which represents a metal-binding protein.

In embodiments where metal-binding polypeptides are selected as indirect agents, it can be advantageous if the polypeptide binds to one or a plurality of metals which possess signal generating properties. Metals with unpaired electrons in the Dorf orbitals may be used, such as, for example, Fe, Co, Mn, Ni, Gd etc., wherein especially Fe is available in high physiological concentrations in organisms. Such agents may form metal-rich aggregates, for example crystalline aggregates, whose diameters are larger than 10 picometers, preferably larger than 100 picometers, 1 nm, 10 nm or specially preferred larger than 100 nm.

Also, metal-binding compounds which have sub-nanomolar affinities with dissociation constants of less than 10-15 M, 10-2 M or smaller may be used to impart functionality for the implant. Typical polypeptides or metal-binding proteins are lactoferrin, ferritin, or other dimetallocarboxylate proteins, or so-called metal catchers with siderophoric groups, such as haemoglobin. A possible method for preparation of such signal generating agents, their selection and the possible direct or indirect agents which are producible in vivo and are suitable as signal generating agents is disclosed in WO 03/075747.

Another group of signal generating agents can be photophysically signal producing agents which consist of dyestuff-peptide-conjugates. Such dyestuff-peptide-conjugates can provide a wide spectrum of absorption maxima, for example polymethin dyestuffs, such as cyanine-, merocyanine-, oxonol- and squarilium dyestuffs. From the class of the polymethin dyestuffs the cyanine dyestuffs, e.g. the indole structure based indocarbo-, indodicarbo- and indotricarbocyanines, can be suitable. Such dyestuffs can be substituted with suitable linking agents and can be functionalized with other groups as desired, see also DE 19917713.

The signal generating agents can be further functionalized as desired. The functionalization by means of so-called “Targeting” groups is meant to include functional chemical compounds which link the signal generating agent or its specifically available form (encapsulation, micelles, micro spheres, vectors etc.) to a specific functional location, or to a determined cell type, tissue type or other desired target structures. Targeting groups can permit the accumulation of signal-producing agents in or at specific target structures. Therefore, the targeting groups can be selected from such substances, which are principally suitable to provide a purposeful enrichment of the signal-generating agents in their specifically available form by physical, chemical or biological routes or combinations thereof. Useful targeting groups can therefore include antibodies, cell receptor ligands, hormones, lipids, sugars, dextrane, alcohols, bile acids, fatty acids, amino acids, peptides and nucleic acids, which can be chemically or physically attached to signal-generating agents, in order to link the signal-generating agents into/onto a specifically desired structure. Exemplary targeting groups may include those which enrich signal-generating agents in/on a tissue type or on surfaces of cells. Here, it may not be necessary for the function that the signal generating agent be taken up into the cytoplasm of the cells. Peptides can be targeting groups, for example, chemotactic peptides that are used to visualize inflammation reactions in tissues by means of signal generating agents; see also WO 97/14443.

Antibodies can be used, including antibody fragments, Fab, Fab2, Single Chain Antibodies (for example Fv), chimerical antibodies, moreover antibody-like substances, for example so-called anticalines, wherein it may not be important whether the antibodies are modified after preparation, recombinants are produced or whether they are human or non-human antibodies. Humanized or human antibodies may be used, such as chimerical immunoglobulines, immunoglobulin chains or fragments (such as Fv, Fab, Fab′, F(ab′)2 or other antigen-binding subsequences of antibodies, which may partly contain sequences of non-human antibodies; humanized antibodies may include human immunoglobulines (receptor or recipient antibody), in which groups of a CDR (Complementary Determining Region) of the receptor are replaced through groups of a CDR of a non-human (spender or donor antibody), wherein the spender species, for example, mouse, rabbit or other has appropriate specificity, affinity, and capacity for the binding of target antigens. In a few forms the Fv framework groups of the human immunglobulines are replaced by means of corresponding non-human groups. Humanized antibodies can moreover contain groups which either do not occur in either the CDR or Fv framework sequence of the spender or the recipient. Humanized antibodies essentially comprise substantially at least one or preferably two variable domains, in which all or substantial components of the CDR components of the CDR regions or Fv framework sequences correspond with those of the non-human immunoglobulin, and all or substantial components of the FR regions correspond with a human consensus-sequence. Targeting groups can also include hetero-conjugated antibodies. The functions of the selected antibodies or peptides include cell surface markers or molecules, particularly of cancer cells, wherein here a large number of known surface structures are known, such as HER2, VEGF, CA15-3, CA 549, CA 27.29, CA 19, CA 50, CA242, MCA, CA125, DE-PAN-2, etc.

Moreover, targeting groups may contain the functional binding sites of ligands which are suitable for binding to any desired cell receptors. Examples of target receptors include receptors of the group of insulin receptors, insulin-like as growth factor receptor (e IGF-1 and IGF-2), growth hormone receptor, glucose transporters (particularly GLUT 4 receptor), transferrin receptor (transferrin), Epidermal Growth Factor receptor (EGF), low density lipoprotein receptor, high density lipoprotein receptor, leptin receptor, oestrogen receptor; interleukin receptors including IL-1, IL-2, IL-3, IL-4, IL-5, IL-6, IL-7, IL-8, IL-9, IL-11, IL-12, IL-13, IL-15, and IL-17 receptor, VEGF receptor (VEGF), PDGF receptor (PDGF), Transforming Growth Factor receptor (including TGF-[alpha] and TGF-[beta]), EPO receptor (EPO), TPO receptor (TPO), ciliary neurotrophic factor receptor, prolactin receptor, and T-cell receptors.

Also, hormone receptors may be used, especially for hormones such as steroidal hormones or protein- or peptide-based hormones, for example, epinephrines, thyroxines, oxytocine, insulin, thyroid-stimulating hormone, calcitonine, chorionic gonadotropine, corticotropine, follicle stimulating hormone, glucagons, leuteinizing hormone, lipotropine, melanocyte-stimulating hormone, norepinephrines, parathyroid hormone, Thyroid-Stimulating Hormone (TSH), vasopressin's, encephalin, serotonin, estradiol, progesterone, testosterone, cortisone, and glucocorticoide. Receptor ligands include those which are on the cell surface receptors of hormones, lipids, proteins, glycol proteins, signal transducers, growth factors, cytokine, and other bio molecules. Moreover, targeting groups can be selected from carbohydrates with the general formula: Cx(H2O)y, wherein monosaccharides, disaccharides and oligo—as well as polysaccharides are also included, as well as other polymers which consist of sugar molecules containing glycosidic bonds. Carbohydrates may include those in which all or parts of the carbohydrate components contain glycosylated proteins, including the monomers and oligomers of galactose, mannose, fructose, galactosamine, glucosamine, glucose, sialic acid, and the glycosylated components, which make possible the binding to specific receptors, especially cell surface receptors. Other useful carbohydrates include monomers and polymers of glucose, ribose, lactose, raffinose, fructose and other biologically occurring carbohydrates especially polysaccharides, for example, arabinogalactan, gum Arabica, mannan etc., which are suitable for introducing signal generating agents into cells, such as described in U.S. Pat. No. 5,554,386.

Furthermore, targeting groups can include lipids, fats, fatty oils, waxes, phospholipids, glycolipids, terpenes, fatty acids and glycerides, and triglycerides, or eicosanoides, steroids, sterols, suitable compounds of which can also be hormones such as prostaglandins, opiates and cholesterol etc. The functional groups can be selected as the targeting group, which possess inhibiting properties, such as, for example enzyme inhibitors, preferably those which link signal generating agents into/onto enzymes.

Targeting groups can also include functional compounds which enable internalization or incorporation of signal generating agents in the cells, especially in the cytoplasm or in specific cell compartments or organelles, such as, for example, the cell nucleus. For example, such a targeting group may contain all or parts of HIV-1 tat-proteins, their analogues and derivatized or functionally similar proteins, and in this way allows an especially rapid uptake of substances into the cells. As an example, refer to Fawell et al., PNAS USA 91:664 (1994); Frankel et al., Cell 55:1189,(1988); Savion et al., J. Biol. Chem. 256:1149 (1981); Derossi et al., J. Biol. Chem. 269:10444 (1994); and Baldin et al., EMBO J. 9:1511 (1990).

Targeting groups can further include the so-called Nuclear Localisation Signal (NLS), which include positively charged (basic) domains which bind to specifically targeted structures of cell nuclei. Numerous NLS and their amino acid sequences are known including single basic NLS such as that of the SV40 (monkey virus) large T Antigen (pro Lys Lys Lys Arg Lys Val), Kalderon (1984), et al., Cell, 39:499-509), the teinoic acid receptor-[beta] nuclear localization signal (ARRRRP); NFKB p50 (EEVQRKRQKL; Ghosh et al., Cell 62:1019 (1990); NFKB p65 (EEKRKRTYE; Nolan et al., Cell 64:961 (1991), as well as others (see, for example, Boulikas, J. Cell. Biochem. 55(1):32-58 (1994), and double basic NLS's such as, for example, xenopus (African clawed toad) proteins, nucleoplasmin (Ala Val Lys Arg Pro Ala Ala Thr Lys Lys Ala Gly Gln Ala Lys Lys Lys Lys Leu Asp), Dingwall, et al., Cell, 30:449-458, 1982 and Dingwall, et al., J. Cell Biol., 107:641-849, 1988. Numerous localization studies have shown that NLSs that are built into synthetic peptides which normally do not address the cell nucleus or were coupled to reporter proteins, lead to an enrichment of such proteins and peptides in cell nuclei. Exemplary references are made to Dingwall, and Laskey, Ann, Rev. Cell Biol., 2:367-390, 1986; Bonnerot, et al., Proc. Natl. Acad. Sci. USA, 84:6795-6799, 1987; Galileo, et al., Proc. Natl. Acad. Sci. USA, 87:458-462, 1990. Targeting groups for the hepatobiliary system may be selected, as described in U.S. Pat. Nos. 5,573,752 and 5,582,814.

In exemplary embodiments, the implant comprises absorptive agents, e.g. to remove compounds from body fluids. Suitable absorptive agents include chelating agents such as penicillamine, methylene tetramine dihydrochloride, EDTA, DMSA or deferoxamine mesylate, any other appropriate chemical modification, antibodies, and microbeads or other materials containing cross linked reagents for absorption of drugs, toxins or other agents.

According to exemplary embodiments of the present invention, functional modification can be achieved by incorporating at least one therapeutically active agent, diagnostic active agent or absorptive agent partially or completely into the polymer particles or into or onto the implant structure. Incorporation may be carried out by any suitable means, such as impregnating, dip-coating, spray coating or the like. The therapeutically active agent, diagnostic agent or absorptive agent may be provided in an appropriate solvent, optionally using additives. The loading of these agents may be carried out under atmospheric, sub-atmospheric pressure or under vacuum. Alternatively, loading may be carried out under high pressure. Incorporation of the therapeutically active agent, diagnostic agent and/or absorptive agent may be carried out by applying an electrical charge to the implant or exposing at least a portion of the implant to a gaseous material including the gaseous or vapor phase of the solvent in which an agent is dissolved or other gases that have a high degree of solubility in the loading solvent. In exemplary embodiments the therapeutically active agents, diagnostic agents or absorptive agents are provided in the polymer particles which serve as a carrier therefor, and which are embedded in the matrix of the metal-based particles of the implant.

Functional modification can also be achieved by selecting the particles appropriately with regard to their biochemical, physical and biological properties. One exemplary embodiment includes the use of x-ray absorptive particles such as tantalum, tungsten etc. as at least a part of the metal-based particles. In other exemplary embodiments, ferromagnetic metal-based particles may be used to achieve visibility in MRI imaging.

Functional modification can also be implemented by adding therapeutically active agents, diagnostic and/or absorptive agents partially or completely to the surface of the inventive implant, for example in a coating

In other embodiments, the therapeutically active agents, diagnostic and/or absorptive agents can be added by introducing them encapsulated, preferably encapsulated in polymeric shells, into the implant body. In these embodiments, the agents represent the polymer particles and the encapsulating material is selected from materials as defined above for the biodegradable polymer particles that allow eluting of the active ingredients by partially or completely dissolving the encapsulating material in physiological fluids.

Further functional modification can be achieved by adding, partially or completely incorporating a material that alters and modulates, hereinafter referred to as altering and modulating material, the availability, function or release of a therapeutically active agent, diagnostic and/or absorptive agents. The altering and modulating material may include a diffusion barrier or a biodegradable material or a polymer or hydrogel. In some exemplary embodiments, the biodegradable polymer particles may further provide a combination of different therapeutically active agents, diagnostic and/or absorptive agents that can be incorporated into different altering and modulating materials.

In other embodiments, functional modification can be carried out by an application of a coating of one or more altering and modulating materials onto at least one part of the implant, whereby the polymer particles of the device have at least one therapeutically active agent, diagnostic or absorptive agent.

In certain exemplary embodiments, it can be of advantage to coat the implant, or at least a part of the implant, with non-degradable or degradable polymers, optionally containing therapeutically or diagnostically or absorptive agents or any mixture thereof.

In another embodiment, it can be desirable to coat the implant on the outer surface or inner surface with a coating to enhance engraftment or biocompatibility. Such coatings may preferably include carbon coatings, metal carbides, metal nitrides, metal oxides e.g. diamond-like carbon or silicon carbide, or pure metal layers of e.g. titanium, using PVD, Sputter-, CVD or similar vapor deposition methods or ion implantation.

In further embodiments a sol/gel-based coating that can be dissolvable in physiological fluids may be applied to at least a part of the implant, as disclosed e.g. in WO 2006/077256 or WO 2006/082221.

In some exemplary embodiments, it can be desirable to combine two or more different functional modifications as described above to obtain a functional implant.

Using conventional means, the implants of the present invention may be generally produced by exemplary methods as described in the following:

### Production of Slurry A

A slurry can be first produced using metal-based nanoparticles and polymeric particulate materials in the appropriate ratio. If a wetting agent is added, the metal-based particles are mixed with the wetting agent and stirred for a certain period of time, e.g., for approximately 20 minutes. Polymer particles are suspended in a solvent, and added to the metal-based particles. The slurry may then be homogenized using a conventional stirrer.

### Molding of Implants

For molding, a suitable mold is to be used. For example, to prepare discoid implants, a standard cylindrical hollow mold made out of stainless steel can be used, e.g., with an inner diameter of 3 cm and a length of 8 cm. The slurry A is filled into the mold until ⅘ of the volume is filled and compacting is carried out by using a standard floating mold die press to form a green body. Subsequently, a compaction pressure of about 50 MPa is applied for about 100 seconds, then repeating the cycle two further times. The green body then has a discoid type shape with a diameter of 2.8 cm and a height of 4 cm. It can be further dried, for example at room temperature for about 1 hour to produce the final implant.

Typically, the implants formed in accordance with the present embodiment, are mechanically stable due to the high compaction forces. An increase of compaction forces also is correlated to an increase of mechanical stability. Usually, bending strengths and toughness values can be in a range from 1 to 200 MPa (bending strength) and 20 to 500 J/m².

Having thus described in detail several exemplary embodiments of the present invention, it should be understood that the invention described above is not to be limited to particular details set forth in the above description, as many apparent variations thereof are possible without departing from the spirit or scope of the present invention. The detailed description is given by way of example and is not intended to limit the invention solely to the specific embodiments described.

The foregoing applications, and all documents cited therein or during their prosecution (“appln. cited documents”) and all documents cited or referenced in the appln. cited documents, and all documents cited or referenced herein (“herein cited documents”), and all documents cited or referenced in the herein cited documents, together with any manufacturer's instructions, descriptions, product specifications, and product sheets for any products mentioned herein or in any document incorporated by reference herein, are hereby incorporated herein by reference in their entireties, and may be employed in the practice of the invention. Citation or identification of any document in this application is not an admission that such a document is available as prior art to the present invention.

What is claimed is:

1. An at least partially biodegradable implant, comprising:
a plurality of first particles of at least one in-vivo biodegradable organic polymer; and
a plurality of second particles of at least one metal-based material, wherein the first particles are embedded in a matrix of compressed second particles.

2. The implant of claim 1, wherein the implant is formed from a suspension comprising the first particles, the second particles, and a solvent; and wherein the suspension is molded under pressure to form the implant.

3. The implant of claim 1, wherein the first particles include at least one of collagen, albumin, gelatin, hyaluronic acid, starch, cellulose, methyl cellulose, hydroxypropyl cellulose, hydroxypropylmethyl cellulose, carboxymethyl cellulose phthalate, casein, dextran, polysaccharide, fibrinogen, poly(D,L-lactide), poly(D,L-lactide-co-glycolide), poly(glycolide), poly(hydroxybutylate), poly(alkyl carbonate), poly(ortho ester), polyester, poly(hydroxyvaleric acid), polydioxanone, poly(ethylene terephtalate), poly(malic acid), poly(tartronic acid), polyanhydride, polyphosphazene, poly(amino acid), and copolymers thereof.

4. The implant of claim 1, wherein the second metal-based particles include at least one of a metal, a metal alloy, a metal oxide, a metal carbide, a metal nitride, or a metal-containing semiconductor.

5. The implant of claim 4, wherein the second particles have an average particle size in a range from about 0.5 nanometer to 500 micrometer.

6. The implant of claim 1, wherein the average particle size of the first particles is higher than the average particle size of the second particles.

7. The implant of claim 4, wherein the metal based material is biodegradable in-vivo.

8. The implant of claim 7, wherein the metal based material is selected from magnesium or zinc, or an alloy comprising at least one of Mg, Ca, Fe, Zn, Al, W, Ln, Si, or Y.

9. The implant of claim 1, wherein at least one of the first and second particles are selected from spherical particles, dendritic particles, cubes, wires, fibres or tubes.

10. The implant of claim 1, wherein the first particles include at least one active ingredient.

11. The implant of claim 1, wherein the second particles include at least one active ingredient.

12. The implant of claim 10, wherein the active ingredient includes at least one of a pharmacologically active agent, a therapeutically active agent and a diagnostically active agent.

13. The implant of claim 11, wherein the active ingredient includes at least one of a pharmacologically active agent, a therapeutically active agent and a diagnostically active agent.

14. The implant of claim 1, wherein the implant is selected from the group consisting of a vascular endoprosthesis, an intraluminal endoprosthesis, a stent, a coronary stent, a peripheral stent, a surgical, dental or orthopedic implant, an implantable orthopedic fixation aid, an orthopedic bone prosthesis or joint prosthesis, a bone substitute or a vertebral substitute in the thoracic or lumbar region of the spinal column; an artificial heart or a part thereof, an artificial heart valve, a heart pacemaker casing or electrode, a subcutaneous and/or intramuscular implant, an implantable drug-delivery device, a microchip, or implantable surgical needles, screws, nails, clips, staples, or a seed implant.

15. A method for producing an at least partially biodegradable implant, comprising:
providing a suspension comprising a plurality of first particles of at least one in-vivo biodegradable organic polymer, a plurality of second particles of at least one metal-based material; and at least one solvent, wherein the first and second particles are substantially insoluble in the solvent; and
molding the suspension to form an implant comprising the first particles embedded in a matrix of compressed second particles.

16. The method of claim 15, wherein the suspension is molded by at least one of compacting, injection molding, uniaxial or biaxial pressing, isostatic pressing, slip casting, or extrusion molding operations.

17. The method of claim 15, wherein the suspension comprises the first and second particles in a volume ratio from about 30:1 to 1:30.

18. The method of claims 15, wherein the combined weight of the first and second particles in the suspension amount to more than 50 wt-% of the suspension in total.

19. The method of claim 15, wherein the suspension is a paste.

20. The method of claim 15, wherein the suspension comprises at least one further additive selected from dispersants or surfactants.

21. The method of claim 15, wherein the molding includes compaction pressures in the range of from about 6,890 kPa (1,000 psi) to about 138,000 kPa (20,000 psi).

22. The method of claim 15, wherein the molding includes compaction times in the range of from about 1 second to about 6000 seconds.
